(19) United States
(12) Patent Application Publication
(10) Pub. No.: US 2023/0338060 A1
(43) Pub. Date: Oct. 26, 2023

(54) Device and Method for Treating Osteonecrosis

(71) Applicant: Texas Scottish Rite Hospital for Children, Dallas, TX (US)

(72) Inventors: Harry K.W. Kim, Dallas, TX (US); Chi Ma, Sunnyvale, TX (US)

(21) Appl. No.: 18/334,212

(22) Filed: Jun. 13, 2023

Related U.S. Application Data
(63) Continuation-in-part of application No. 16/945,270, filed on Jul. 31, 2020.
Application No. 16/945,270 is a continuation of application No. 16/936,084, filed on Jul. 22, 2020.
Application No. 16/936,084 is a continuation of application No. 15/956,245, filed on Apr. 18, 2018, now Pat. No. 10,765,453.
Application No. 15/956,245 is a continuation-in-part of application No. 15/490,595, filed on Apr. 18, 2017, now Pat. No. 10,758,253.

(51) Int. Cl. A61B 17/34; A61B 17/17; A61B 17/16; A61M 3/02

(52) U.S. Cl. CPC A61B 17/3472; A61B 17/17; A61B 17/1622; A61B 17/1714; A61B 17/1725; A61B 17/1764; A61B 17/1668; A61B 17/1664; A61M 3/0204; A61B 17/1697; A61M 3/0212; A61B 17/1721; A61M 3/022; A61B 2217/007; A61B 2090/062

(57) ABSTRACT

Provided herein are methods and compositions for treating bone osteonecrosis comprising: at least one of a Gelatin tyramine (GT), a gelatin-heparin tyramine (GHT), a gelatin methacryloyl (GelMa), a gelatin acryloyl (GelAC), collagen, or a hydrogel-forming peptide that can be formed ex vivo or in situ for the treatment of bone osteonecrosis; at least one bone growth promoting agent or a macrophage depletion reagent; and optionally one or more pharmaceutically acceptable carriers.

### CROSS-REFERENCE TO RELATED APPLICATIONS

This application is a continuation-in-part application of U.S. patent application Ser. No. 16/945,270 filed on Jul. 31, 2020, which is a continuation application of U.S. patent application Ser. No. 16/936,084 filed on Jul. 22, 2020, which is a continuation of U.S. patent application Ser. No. 15/956,245 filed Apr. 18, 2018, now U.S. Pat. No. 10,765,453 issued on Sep. 8, 2020, which is a continuation-in-part application of U.S. patent application Ser. No. 15/490,595 filed on Apr. 18, 2017, now U.S. Pat. No. 10,758,253 issued on Sep. 1, 2020.

### STATEMENT OF FEDERALLY FUNDED RESEARCH

This invention was made with government support under 1R01AR078311 awarded by the National Institutes of Health. The government has certain rights in the invention.

### TECHNICAL FIELD OF THE INVENTION

The present invention relates in general to the field of osteonecrosis, and more particularly, to a novel device and method for treating osteonecrosis, such as, Legg-Calve-Perthes disease.

### BACKGROUND OF THE INVENTION

Without limiting the scope of the invention, its background is described in connection with tissue regeneration.

One such method is taught in U.S. Pat. No. 9,138,317, issued to McGee, entitled “Conduits for enhancing tissue regeneration”, which is said to teach apparatuses, systems, and methods for enhancing bone or soft tissue regeneration. For example, a conduit, having one or more segments, can originate at a tissue regeneration site and can have a first opening to promote physiological signals to enter the conduit and transit to a second opening that penetrates a histologically rich source of multipotent mesenchymal cells, promoting the multipotent mesenchymal cells to produce tissue regeneration response products, the response products transiting through the second opening to egress at the first opening of the conduit, and promoting tissue regeneration at the tissue regeneration site.

Another such method is taught in U.S. Pat. No. 8,382,762, issued to Brannon, entitled “Endoscopic bone debridement”, which is said to teach an osteoendoscopic cylinder for tamponading bleeding along a longitudinal canal surface of an osteocentral canal of a femoral neck so as to allow endoscopic visualization of a segment of osteonecrotic bone within a femoral head. The osteoendoscopic cylinder is of a dimension adapted to receive an endoscope therein and includes an inner visual surface and an outer bony contact surface. An orientation mark along the inner visual surface is of a size and dimension to ensure a first visualization thereof with the endoscope.

Despite these advances, a need remains for compositions and methods for treating osteonecrosis, such as, Legg-Calve-Perthes disease.

### SUMMARY OF THE INVENTION

As embodied and broadly described herein, an aspect of the present disclosure relates to a method of treating bone osteonecrosis comprising: preparing a bone growth-promoting hydrogel comprising one or more bone growth-promoting agents in a hydrogel, the hydrogel comprising: at least one of gelatin tyramine (GT), gelatin-heparin tyramine (GHT), gelatin methacryloyl (GelMa), gelatin acryloyl (GelAC), or collagen, or a hydrogel-forming peptide; at least one bone growth-promoting agent or a macrophage depletion reagent; and optionally one or more pharmaceutically acceptable carriers; and injecting the bone growth-promoting hydrogel into one or more openings in a bone, wherein the bone growth-promoting hydrogel treats bone osteonecrosis. In one aspect, the growth-promoting agent or macrophage depletion reagent is selected from bone morphogenic protein 2 (BMP-2), bone morphogenic protein 7 (BMP-7), Vascular endothelial growth factor (VEGF), clodronate, or Clodrosome. In another aspect, the composition is a biocompatible isotonic fluid and optionally comprises biocompatible detergents, biocompatible surfactants, biocompatible alcohols, antibiotics, preservatives, or combinations thereof, wherein the washing fluid cleans at least 50, 60, 70, 75, 80, 85, or 90% of the volume within the bone. In another aspect, the method further comprises at least one of autologous bone marrow, autologous bone stem cells, bone graft material, bone void filler, cancellous bone graft or fragments, osteoconductive material, osteoproliferative material, osteoinductive material, or bone material infused collagen matrix. In another aspect, the GT is a

Gelatin tyramine hydrogel, a gelatin-heparin tyramine (GHT) hydrogel, or both (1 to 10% (w/v)), which comprises a trace amount of hydrogen peroxide (0.1 mM-10 mM), a horseradish peroxidase enzyme (0.1-10 unit/ml), wherein the bone growth promoting agent or a macrophage depletion reagent retains at least 80% bioactivity, and optionally comprising heparin at (0.5-5% w/v) that is formed ex vivo or in situ. In another aspect, the GelMa is a Gelatin methacryloyl (GelMa) hydrogel, a gelatin acryloyl (GelAC) hydrogel, or both (2 to 10% (w/v)), which comprises a photoinitiator at (0.01-1% w/v) and the hydrogel is formed by ultraviolet light ex vivo or in situ. In another aspect, the collagen is a collagen hydrogel (0.3-3%, w/v) formed by gelation at 37° C. without the addition of crosslinkers that is formed ex vivo or in situ. In another aspect, the bone is a femoral head, a humeral head, a knee condyle, a proximal tibia, or an ankle talus. In another aspect, the osteonecrosis is Legg-Calvé-Perthes Disease, idiopathic (unknown cause), due to corticosteroid, trauma, alcohol, sickle cell disease, or other known causes of osteonecrosis. In another aspect, the method further comprises the step of washing an interior of the bone prior to injecting the bone growth-promoting hydrogel.

As embodied and broadly described herein, an aspect of the present disclosure relates to a composition for treating bone osteonecrosis comprising: at least one of a Gelatin tyramine (GT), a gelatin-heparin tyramine (GHT), a gelatin methacryloyl (GelMa), a gelatin acryloyl (GelAC), collagen, or a hydrogel-forming peptide that is formed ex vivo or in situ for the treatment of bone osteonecrosis and that prevent or reduce leakage of an active agent, wherein the active agent comprises at least one bone growth promoting agent or a macrophage depletion reagent in an amount sufficient to reduce or eliminate bone osteonecrosis; and optionally one or more pharmaceutically acceptable carriers, wherein the composition treats bone osteonecrosis. In one aspect, the growth promoting agent or macrophage depletion reagent is selected from bone morphogenic protein 2 (BMP-2), bone morphogenic protein 7 (BMP-7), Vascular endothelial growth factor (VEGF), clodronate, or Clodrosome. In another aspect, the composition is a biocompatible isotonic fluid and optionally comprises biocompatible detergents, biocompatible surfactants, biocompatible alcohols, antibiotics, preservatives, or combinations thereof, wherein the washing fluid cleans at least 50, 60, 70, 75, 80, 85, or 90% of the volume within the bone. In another aspect, the composition further comprises at least one of autologous bone marrow, autologous bone stem cells, bone graft material, bone void filler, cancellous bone graft or fragments, osteoconductive material, osteoproliferative material, osteoinductive material, or bone material infused collagen matrix. In another aspect, the GT is a Gelatin tyramine hydrogel, a gelatin-heparin tyramine (GHT) hydrogel, or both, which comprises a trace amount of hydrogen peroxide (0.1 mM-10 mM), a horseradish peroxidase enzyme (0.1-10 unit/ml), wherein the bone growth promoting agent or a macrophage depletion reagent retains at least 80% bioactivity, and optionally comprising heparin at (0.5-5% w/v) that is formed ex vivo or in situ. In another aspect, the GelMa is a Gelatin methacryloyl (GelMa) hydrogel, a gelatin acryloyl (GelAC) hydrogel, or both, which comprises a photoinitiator at (0.01-1% w/v) and the hydrogel is formed by ultraviolet light ex vivo or in situ. In another aspect, the collagen is a collagen hydrogel (0.3-3%, w/v) formed by gelation at 37° C. without the addition of crosslinkers that is formed ex vivo or in situ. In another aspect, the hydrogel forming peptide is synthetic peptide-RADA16 (0.25%-2.5%, w/v) is a thermosensitive hydrogel that is formed ex vivo or in situ. In another aspect, the composition is adapted to inject into a femoral head, a humeral head, a knee condyle, a proximal tibia, or an ankle talus. In another aspect, the composition is adapted for injection into an osteonecrosis is Legg-Calvé-Perthes Disease, idiopathic (unknown cause), due to corticosteroid, trauma, alcohol, sickle cell disease, or other known causes of osteonecrosis.

As embodied and broadly described herein, an aspect of the present disclosure relates to a method of making a composition for injection into osteonecrotic tissue comprising: mixing at least one of a Gelatin tyramine (GT), a gelatin-heparin tyramine (GHT), a gelatin methacryloyl (GelMa), a gelatin acryloyl (GelAC), collagen, or a hydrogel-forming peptide in an amount sufficient to form a hydrogel, and one or more bone growth promoting agents or macrophage depletion reagents in an amount effective to induce bone growth in a femoral head, a humeral head, a knee condyle, a proximal tibia, or an ankle talus; and optionally one or more pharmaceutically acceptable carriers, wherein the composition treats bone osteonecrosis. In one aspect, the growth promoting agent or macrophage depletion reagent is selected from bone morphogenic protein 2 (BMP-2), bone morphogenic protein 7 (BMP-7), Vascular endothelial growth factor (VEGF), clodronate, or Clodrosome. In another aspect, the composition is a biocompatible isotonic fluid and optionally comprises biocompatible detergents, biocompatible surfactants, biocompatible alcohols, antibiotics, preservatives, or combinations thereof, wherein the washing fluid cleans at least 50, 60, 70, 75, 80, 85, or 90% of the volume within the bone. In another aspect, the method further comprises at least one of autologous bone marrow, autologous bone stem cells, bone graft material, bone void filler, cancellous bone graft or fragments, osteoconductive material, osteoproliferative material, osteoinductive material, or bone material infused collagen matrix. In another aspect, the GT is a Gelatin tyramine hydrogel, a gelatin-heparin tyramine (GHT) hydrogel, or both, which comprises a trace amount of hydrogen peroxide (0.1 mM-10 mM), a horseradish peroxidase enzyme (0.1-10 unit/ml), wherein the bone growth promoting agent or a macrophage depletion reagent retains at least 80% bioactivity, and optionally comprising heparin at (0.5-5% w/v) that is formed ex vivo or in situ. In another aspect, the GelMa is a Gelatin methacryloyl (GelMa) hydrogel, a gelatin acryloyl (GelAC) hydrogel, or both, which comprises a photoinitiator at (0.01-1% w/v) and the hydrogel is formed by ultraviolet light ex vivo or in situ. In another aspect, the collagen is a collagen hydrogel (0.3-3%, w/v) formed by gelation at 37° C. without the addition of crosslinkers that is formed ex vivo or in situ. In another aspect, the hydrogel-forming peptide is synthetic peptide-RADA16 (0.25%-2.5%, w/v) is a thermosensitive hydrogel that is formed ex vivo or in situ.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B are illustrations of the tissue engineering design for the treatment of LCPD. (FIG. 1A) The synthetic route of the gelatin-heparin-tyramine hydrogel precursor, the preparation of BMP2-hydrogel, and in vitro characterizations; (FIG. 1B) The flowchart of the in vivo experimental design and characterizations of the BMP2-hydrogel treatment on the porcine model of LCPD.

FIGS. 2A to 2D show that the GHT hydrogel can retain and preserve long-term bioactivities of BMP2. (FIG. 2A) The graph showing the cumulative release profile of BMP2 from GHT hydrogel (0.75 mg BMP2 per ml of hydrogel); (FIG. 2B) The bar graph showing the total amount of detected BMP2 from the GHT hydrogel; (FIG. 2C) The bar graph showing the relative gene expression by pig bone marrow mesenchymal cells (pBMSCs) cultured for 5 days in growth medium (GM), osteogenic medium (OM), OM supplemented with BMP2 (40 ng/ml), or OM supplemented with released BMP2 (40 ng/ml) from the hydrogel (BMP2-R); (FIG. 2D) Alizarin Red staining of the pBMSCs that were cultured for 11 days in GM, OM, OM+BMP2 (40 ng/ml), and OM+BMP2-R (40 ng/ml). * represents p<0.05, when comparing with GM group; # represents p<0.05, when comparing with OM group.

FIGS. 3A to 3F shows that the injection of the hydrogel carrier produces a broad distribution in the femoral head without leakage. (FIG. 3A). The pictures show the preparation for testing leakage ex vivo. FIG. 3A1 is the blue dye-labeled saline, and FIG. 3A2 is the blue dye-labeled hydrogel. FIG. 3A3 is the apparatus setup for testing the injection of hydrogel ex vivo, including the cadaveric porcine femur, 3D printed guide, and three drilling needles; FIG. 3A4 and FIG. 3A5 are X-ray images depicting the sagittal and coronal views post-drilling. (FIG. 3B). The digital pictures show the leakage after the saline injection but not the hydrogel injection. FIG. 3B1 shows the blue dye-labeled saline leaking out from the porcine femoral head after injection; FIG. 3B2 shows no hydrogel leaking out from the porcine femoral head after injection; FIG. 3B3 & FIG. 3B4 are the images from tissue-cleared samples; FIG. 3B3 shows the backflow of saline to the metaphysis, whereas FIG. 3B4 shows no backflow of hydrogel to the metaphysis. (FIG. 3C) Representative X-ray and 2D μCT images demonstrate the leakage that occurred when 0.6 ml, 1.2 ml, and 1.5 ml of saline was injected (the red arrows show the leaked saline), and FIG. 3D) The graph shows the percentage leakage of saline; (FIG. E) X-ray and 2D μCT images show no leakage when 0.6 ml, 1.2 ml, and 1.5 ml of hydrogel was injected; (FIG. 3F) The graph shows the distribution of saline and hydrogel in the femoral head after injection.

FIGS. 4A to 4C shows that BMP2 hydrogel-treated femoral head produced a semi-spherical shape with no bone defect. (FIG. 4A) Representative pictures of the bisected femoral heads from normal, saline wash, and BMP2-hydrogel treatment groups; (FIG. 4B) Representative X-ray images of the femoral heads from the normal, saline wash, and BMP2-hydrogel treatment groups; (FIG. 4C) Bar graph showing the mean EQ value of the femoral heads from the normal, saline wash, and BMP2-hydrogel groups. White asterisks indicate areas with large bone voids occupied by dense fibrous tissue.

FIGS. 5A to 5E BMP2 hydrogel treatment repaired the subchondral and trabecular bones of femoral head (FIG. 5A-5C) Representative H&E staining images of the whole femoral heads of normal, saline wash, and BMP2-hydrogel groups; (FIG. 5A1-FIG. 5C1) Magnified images of the subchondral regions of normal, saline wash, and BMP2-hydrogel groups; (FIG. 5A2-FIG. 5C2) Magnified images of the epiphyseal trabeculae of normal, saline wash, and BMP2-hydrogel groups; (FIG. 5D) Percentage of the subchondral region with restored endochondral ossification from the normal, saline wash, and BMP2-hydrogel groups; (FIG. 5E) Percentage of empty lacunae from the normal, saline wash, and BMP2-hydrogel groups. Black dash lines show the normal subchondral bone. Red dash lines show the abnormal subchondral bone; Red arrows show the osteoclasts; Yellow arrows show the empty lacunae. * represent p<0.05; *** represent p<0.001.

FIGS. 6A to 6D show that BMP2 hydrogel treatment accelerated epiphyseal bone regeneration and remodeling of femoral head. (FIG. 6A) Representative fluorescent microscopic images show the calcein (green) labeled bone surface in the femoral heads of the normal, saline wash, and BMP2-hydrogel groups; (FIG. 6B) Ratio of mineralizing surface to the total tissue area (MS/TA, 1/mm); (FIG. 6C) Representative images of tartrate-resistant acid phosphatase (TRAP) staining show osteoclasts on the trabeculae in the normal, saline wash, and BMP2-hydrogel groups; (FIG. 6D) Number of osteoclasts per bone surface (N.Oc/BS, 1/mm) of the normal, wash, and BMP2-hydrogel groups. * represents p<0.05; **** represents p<0.0001.

FIGS. 7A to 7F shows that BMP2 hydrogel treatment produced a homogeneous bone regeneration of femoral head. FIG. 7A) Representative 3D μ-CT images of femoral heads from the normal, saline wash, and BMP2-hydrogel groups. Bar graphs showing (FIG. 7B) the percentage of bone voids, (FIG. 7C) the percentage of bone volume, (FIG. 7D) the trabecular thickness (Tb.Th), (FIG. 7F) the trabecular separation (Tb.Sp), and (FIG. 7E) the trabecular number (Tb.N) of the normal, saline wash, and BMP2-hydrogel groups. * represents p<0.05; ** represents p<0.01; *** represents p<0.001; **** represents p<0.0001.

FIG. 8 is a flowchart of the in vivo pig experimental design and treatment outcome assessment methods.

FIGS. 9A to 9D show clodrosome (CL) treatment improved the subchondral recovery and preserved the trabeculae of the femoral head. FIG. 9A-FIG. 9C) Representative H&E staining images of the whole femoral heads of normal, NWB, and CL groups. The black dash lines show the normal subchondral region with active endochondral ossification. Red dash lines show the abnormal subchondral region with disruption of endochondral ossification. FIG. 9A1-FIG. 9C1) Magnified images of the subchondral regions of the normal, NWB, and CL groups. FIG. 9A2-FIG. 9C2) Magnified images of the epiphyseal trabeculae of the normal, NWB, and CL groups. The red arrows show the osteoclasts. The yellow arrows show the empty lacunae. FIG. 9D) Percentage of the subchondral region with restored endochondral ossification from the normal, NWB, and CL groups. * represents p<0.05.

FIGS. 10A to 10D show that CL treatment better preserved the semi-spherical shape of the femoral head and decreased areas of bone voids. Representative X-ray images of the femoral heads from the (FIG. 10A) normal, (FIG. 10B) NWB, and (FIG. 10C) CL treatment groups. The white asterisks indicate areas with large bone voids. FIG. 10D) Bar graph showing the EQ value of the femoral heads from the normal, NWB, and CL groups. * represents p<0.05; ** represents p<0.005.

FIGS. 11A to 11H show that CL-treated femoral head preserved the trabecular architectures of the femoral head. FIG. 11A-FIG. 11C) Representative 3D μ-CT images of femoral heads from the (FIG. 11A) normal, (FIG. 11B) NWB, and (FIG. 11C) CL groups. Bar graphs showing FIG. 11D) the percentage of the bone voids volume in the epiphysis, FIG. 11E) the BV/TV, FIG. 11F) the Tb.Th., FIG. 11G) the Tb.Sp., and FIG. 11H) the Tb.N. of the normal, NWB, and CL groups. * represents p<0.05; ** represents p<0.01; *** represents p<0.001; **** represents p<0.0001.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

Legg-Calvé-Perthes disease (LCPD) is a childhood ischemic osteonecrosis of the femoral head (ONFH) that affects 1 in 1200 children¹. It is one of the most serious conditions affecting the pediatric hip joint, especially in teenagers, as over 50% of patients will develop debilitating osteoarthritis despite receiving treatments²⁻⁴. ONFH causes the local death of osteocytes and bone marrow cells due to a disruption of blood supply to the bone, and the subsequent repair process results in necrotic bone resorption and structural deformities⁵. The progression of LCPD leads to hip pain, limited range of motion, and physical disability necessitating a total hip replacement (THR)⁶. However, THR is not an optimal treatment option for young patients due to their high physical demand and longevity.

Current treatments to prevent the progression of LCPD, such as weight-bearing restrictions, bracing, and osteotomies show poor clinical outcomes, especially in teenagers with LCPD^{7,8}. Single-tunnel decompression or multiple epiphyseal drillings are common operative treatments to decompress necrotic bone⁹⁻¹¹. It is believed that decompression can create intraosseous tunnels to facilitate revascularization and repair of the necrotic femoral head. Clinical studies, however, showed that decompression alone has limited treatment efficacy, and the failure rate can reach 50%¹²⁻¹⁴.

Necrotic Bone Washing Technique. The bone washing technique of the present invention involves placement of two or more intra-osseous needles or cannulas into pediatric or adult femoral heads for treatment of avascular necrosis (AVN) or osteonecrosis. This technique permits inflow and outflow of washing solution through the needle(s) or cannula(s) to remove, e.g., dead cell debris, necrotic marrow fat, and/or inflammatory factors. It is demonstrated herein that the removal of dead cell debris, necrotic marrow fat, and/or inflammatory factors from the marrow space significantly improves bone healing and creates space for the injection or infusion of biological therapeutic agents and/or stem cells.

Briefly, two or more intra-osseous needles and/or cannulas are placed 5-15 mm apart depending on the necrotic bone volume and the size of the femoral head. Either trans-articular (through the joint and articular cartilage) or trans-physeal/metaphyseal (starting from region below the greater trochanter) or combination needle placement technique can be used. The needles are most often inserted under fluoroscopic guidance and a specialized needle placement device may be used to facilitate the placement of the needles. The present invention can be used in a wide variety of locations that includes osteonecrosis, e.g., the femoral head, the humeral head, the knee condyle, or the ankle talus.

After the placement of two or more intra-osseous needles and/or cannulas within the necrotic bone, aspiration (negative pressure), injection/infusion (positive pressure), and/or a combination of both, are used to provide a high volume of washing solution to flow through the necrotic femoral head to remove the dead cell debris. A high volume washing of the necrotic bone can be facilitated by using a mechanical device or a pump. The amount of volume required for washing or the termination of the washing technique can be determined by assessing the clarity/turbidity of the outflow solution. Further assessment of the outflow solution can be done by measuring levels of specific inflammatory factors using visual, qualitative and/or quantitative assays.

The needles and/or cannulas will generally have following specifications: (1) 6, 7, 8, 9, 10, 11, 12, 13, 14, 15, or 6-15 gauge in diameter, depending on the size of the femoral head and the bone necrosis; (2) the tip of the needle should be less than 1.5 mm long to avoid unintentional penetration through the femoral head, but can be 8, 9, 10, 12, 15, 20, 25, 30 or more centimeters, e.g., 10 to 30 cm; and/or (3) the needle may have one or more fenestrations near the tip to increase the distribution and collection of bone washing solution.

A wide variety of washing solutions and combinations of solutions are contemplated for use with the present invention. The washing solution can be based on saline, various concentrations of ethanol, and/or include one or more biocompatible detergent and/or surfactants for removing/extracting cell debris, necrotic fat, or the necrotic extracellular matrix. The washing solution may also include antibiotics or other antimicrobial agents. The washing solution may also contain one or more bioactive agents, enzymes, or nanoparticles that facilitate the removal of the necrotic fat and the extracellular matrix in the necrotic bone marrow. The washing solution may also contain drugs or agents that stimulate angiogenesis (for instance by activation of hypoxia inducible factor-1 and vascular endothelial growth factor pathways) or stimulate osteogenesis (for instance through Wnt and/or BMP signaling pathways).

Generally, the temperature of wash solution should be warmed up to the body temperature to be physiological and more effective in removing cell debris.

New Stem Cell or Bone Active Agent Delivery Technique. The bone delivery technique involves placement of two or more intra-osseous needles and/or cannulas into pediatric or adult femoral heads for treatment of avascular necrosis (AVN) or osteonecrosis. This technique can be used with or without first performing the bone washing technique outlined above. The use of two or more intra-osseous delivery needles and/or cannulas improves the local distribution of stem cells or bone active agents in the necrotic femoral head by subdividing the total volume of cells or bone active agents to be injected into multiple sites. The technique also improves the local retention of stem cells or bone active agents by decreasing the backflow pressure, unlike the single needle delivery technique where the total volume of injectant is delivered through a single needle site.

This technique can be used to inject cells or bone active agents alone or in combination with a delivery/carrier agent such as hydrogel or gelatin, which can be chemically designed to improve the retention of stem cells and growth factors such as bone morphogenetic proteins (BMPs).

The use of two or more needles and/or cannulas also permits a one or two-step preparation of the necrotic bone for the delivery of stem cells or bone active agents. In a first step, a bone washing and preparation solution is used to remove the cell debris and to distribute a chemical or catalyst required for a chemical reaction which will improve the local retention of a delivery agent and a growth factor when they are injected or infused in the second step. The needle and/or cannulas specification are same as that for the bone washing technique described above. Alternatively, a wash step is not necessary, and a single step can be used to inject or infuse bone active agents and/or stem cells into the bone, that is, intraosseous. Thus, the user has the option of a bone wash step followed by injection of the bone active agents or can directly inject bone active agents without bone washing.

Four groups of hydrogels below that have the potential for osteonecrosis treatment for two reasons. First, the polymers used in these systems have been used in clinics due to their good compatibility with blood/cell/tissue. For example, collagen and its derivatives have been approved by FDA for dental and orthopedic applications, such as collagen membranes (Bio-Gide, etc.) and demineralized bone matrix (Bio-Oss Collagen, StaGraft etc.). Gelatin, which is denatured collagen, has also been FDA-approved as an ingredient in vaccines and implantable device. Second, the drug loading and hydrogel crosslinking do not affect the bioactivity of the osteogenic factors and can produce an even distribution of the osteogenic factors within the hydrogel.

Gelatin tyramine (GT) and gelatin-heparin tyramine (GHT) hydrogels: the precursor polymers of GT and GHT are modified with phenol groups on the gelatin chain by tyramine. GT (or GHT) can be crosslinked with a trace amount of hydrogen peroxide (0.1 mM-10 mM) using the enzyme, horseradish peroxidase (0.1-10 unit/ml). The crosslinking has a minor effect on the bioactivity of proteins⁵. With the chemically grafting of heparin on the gelatin chain (0.5-5% w/v), the GHT can further improve the bioactivity of the loaded proteins. It was found that the GHT hydrogel system retained over 80% bioactivity of BMP2. BMP2-loaded GT and GHT hydrogels can be used for osteonecrosis treatment.

Gelatin methacryloyl (GelMa) and gelatin acryloyl (GelAC) hydrogels: the precursor polymers of GelMa and GelAC have alkene groups on the chain of gelatin because of the modification of methacrylate and acrylate, respectively. By adding the photoinitiators, such as lithium phenyl (2,4,6-trimethylbenzoyl) phosphinate (LAP) or Irgacure 2959 (0.01-1% w/v) in the GelMa (or GelAC) aqueous solution, GelMa (GelAC) can be cross-linked and gelified under UV exposure. It has been reported that GelMa has been used as a BMP2 carrier^{7,8}. GelMa (or GelAC) hydrogel precursor (2%-10%, w/v)/bioactive factors/initiators can be evenly mixed in aqueous solutions, and the hydrogel can be formed after UV exposure. hydrogel/BMP2 can be used for osteonecrosis treatment. Collagen is thermosensitive in an aqueous solution due to the amino acid sequence of (Pro-Hyp-Gly)ₓ, which forms a triple helix conformation that resembles the protein structure of natural collagens. The gelation of collagen occurs at 37° C. with no addition of crosslinkers. Collagen (0.3-3%, w/v) and BMP2 can be mixed at 4-10° C. in an aqueous solution, and the hydrogel can be formed at 37° C. Collagen/BMP2 can be used for osteonecrosis treatment.

Similar to collagen hydrogel, a synthetic peptide-RADA16, can form thermosensitive hydrogel as well. RADA16 hydrogel (0.25%-2.5%, w/v) showed in vitro that it can be used as a BMP2 carrier for osteonecrosis.²⁸

Bone morphogenetic protein 2 (BMP2) belongs to the transforming growth factor beta family and has strong osteogenic potential¹⁵⁻¹⁷. Exogenous recombinant human BMP2 has been approved for tibial non-union fractures and spinal fusion¹⁸. It is reported that local administration of BMP2 can significantly increase new bone formation and decrease the risk of femoral head deformation¹⁹⁻²¹. Saline has been used as a carrier for local BMP2 injection to treat ONFH. However, using saline as the carrier was associated with a high incidence of heterotopic ossification (HO) within the hip capsule²²⁻²⁴ as it has little ability to confine the leaking of BMP2 during the injection. The leakage also reduces the local dose and distribution at the target site and results in incomplete bone regeneration and heterogeneous bone repair²⁰.

Bone morphogenetic protein 7 (BMP-7) is a potent osteogenic factor and has been used in the treatment of tibial non-union¹¹. It is reported that BMP-7 reverses fibrosis through the reduction in monocyte infiltration into inflamed tissue¹².

Vascular endothelial growth factor (VEGF) is a potent angiogenic factor and is an essential growth factor for vascular endothelial cells. Osteogenesis and angiogenesis are two closely correlated processes during bone growth, development, remodeling, and repair. It is reported that VEGF could promote endochondral bone and intramembrane bone formation¹³.

Clodrosome is a macrophage depletion reagent. It is composed of clodronate, a type of bisphosphonate, that is encapsulated within the liposome. Osteoclasts play a major role in bone resorption following osteonecrosis. Clodrosome can inhibit osteoclastogenesis and osteoclast activity via the depletion of macrophages which are the precursor cells of osteoclasts. Local administration of Clodrosome was found to promote subchondral repair and protect the trabecular bone structure and morphology following osteonecrosis in the piglet model, preventing the femoral head from collapsing.

Orthopaedic biomaterials, such as granular or sponge bone grafts, have been tested as BMP2 carriers for the treatment of ONFH^{19,25}. These modalities can be compacted into drilled tunnels. With such application, osteoinduction only occurred within the region of the drilled tunnels, and not in the rest of the necrotic bone. Injectable bone cement (polymethylmethacrylate, PMMA) exhibited a broad distribution within the bone after injection by spreading into the trabecular space. It also provided high mechanical support after setting. However, the setting of PMMA generates a high local temperature which inactivates BMP2²⁶. To the inventors' best knowledge, there are no clinically available tissue engineering strategies that can be used as an effective treatment of ONFH.

An ideal tissue engineering strategy for the treatment of ONFH requires delivering effective bioactive factors (such as BMP2) to the necrotic head with broad distribution and provides sustained osteoinduction. Injectable hydrogels are able to adapt to different shapes in realtime and are widely used as minimally invasive cell/drug carriers in bone tissue regeneration²⁷. In the inventors' previous study, the distribution of a pre-clinical hydrogel was tested on an ex vivo ONFH model, which demonstrated that the hydrogel was injected into the trabecular space beyond the drilled tunnels²⁸. Furthermore, the inventors had previously developed a bone wash technique to remove necrotic bone marrow and debris from the necrotic area²⁹. It was shown that a local necrotic bone wash may better facilitate the delivery of injectable biomaterials. Moreover, the inventors had previously used a heparin-modified gelatin molecule that could specifically bind to bioactive proteins of the transforming growth factor beta family, involving BMP2 and VEGF. This binding effect prevented the bioactive proteins from denaturation and proteolytic degradation^{30,31}.

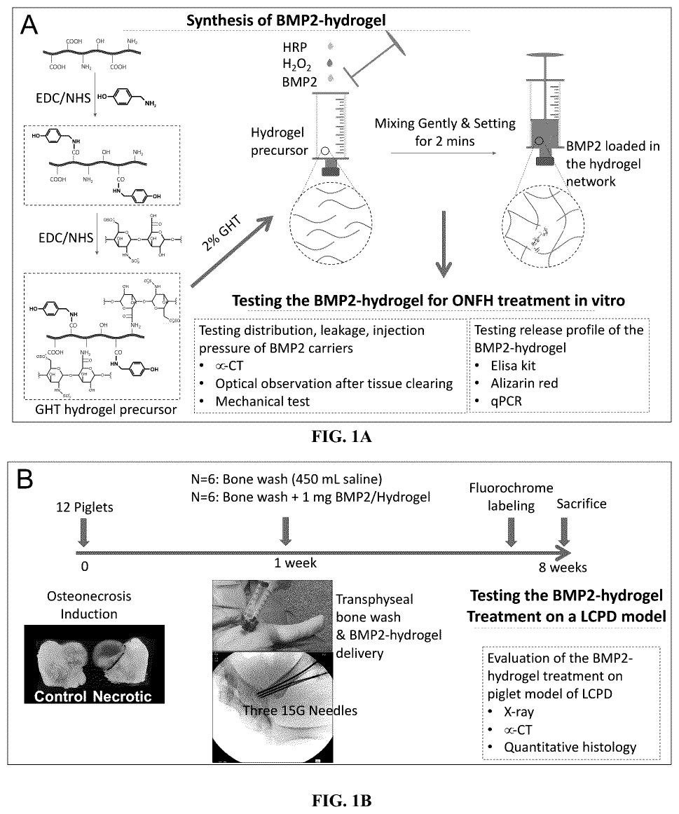

In this study, the inventors applying the bone wash technique and then injecting a BMP2-loaded hydrogel to achieve a broad distribution of the bioactive BMP2 within the necrotic head, further producing an advanced bone regeneration in the context of ONFH. To test the hypothesis, the inventors synthesized a gelatin-heparin-tyramine (GHT) hydrogel as the BMP2 carrier³². The release profile of the BMP2-loaded hydrogel was examined over five weeks in vitro. The osteogenic bioactivity of the released BMP2 was tested in vitro by assays of qPCR and Alizarin red. The inventors examined the leakage following the injection of the hydrogel and saline via optical observations after tissue clearing. The inventors investigated the distribution of the injected hydrogel and saline via loading radiocontrast and micro-CT (μCT) scanning. (FIG. 1A). After that, the inventors systemically assessed the in vivo effects of the BMP2-hydrogel treatment using a piglet model of LCPD. Visual, radiographic, histologic, histomorphometric, and μCT assessments were performed. (FIG. 1B).

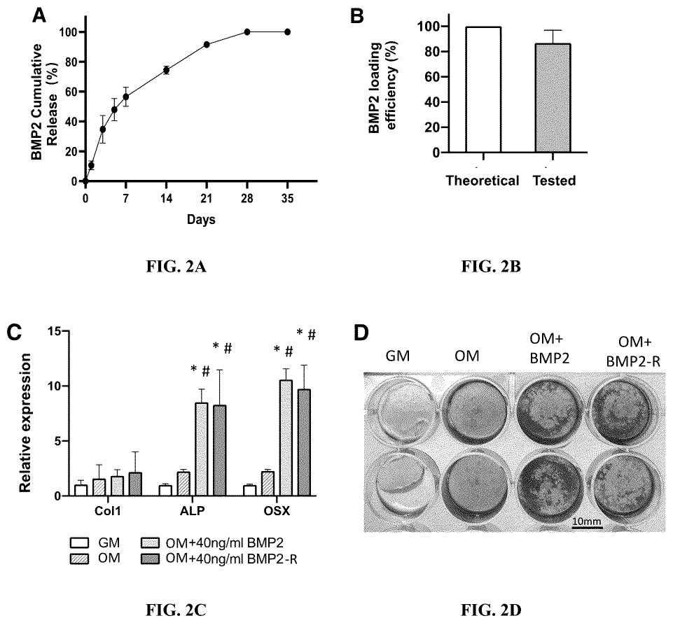

In vitro release of BMP2 from the BMP2-hydrogel. The BMP2 release, loading and release efficiency, and the bioactivity of the released BMP2 were tested in vitro. A high dose of BMP2 (0.75 mg/ml) was mixed with hydrogel precursor, H₂O₂, and horseradish peroxidase (HRP) for the BMP2-hydrogel preparation. As depicted in FIG. 2A, the system only exhibited a 10% burst release of BMP2 in the first 24 hours. Near 60% of BMP2 was released over the first week. During the second and third weeks, a near-linear release profile was observed. More than 97% of BMP2 was released by the end of the fourth week. At 5 weeks, 88% of the theoretically loaded BMP2 was detected by an ELISA kit. (FIG. 2B) These data demonstrate that the GHT hydrogel can sustain BMP2 for four weeks, and the efficiency of BMP2 loading and release is high.

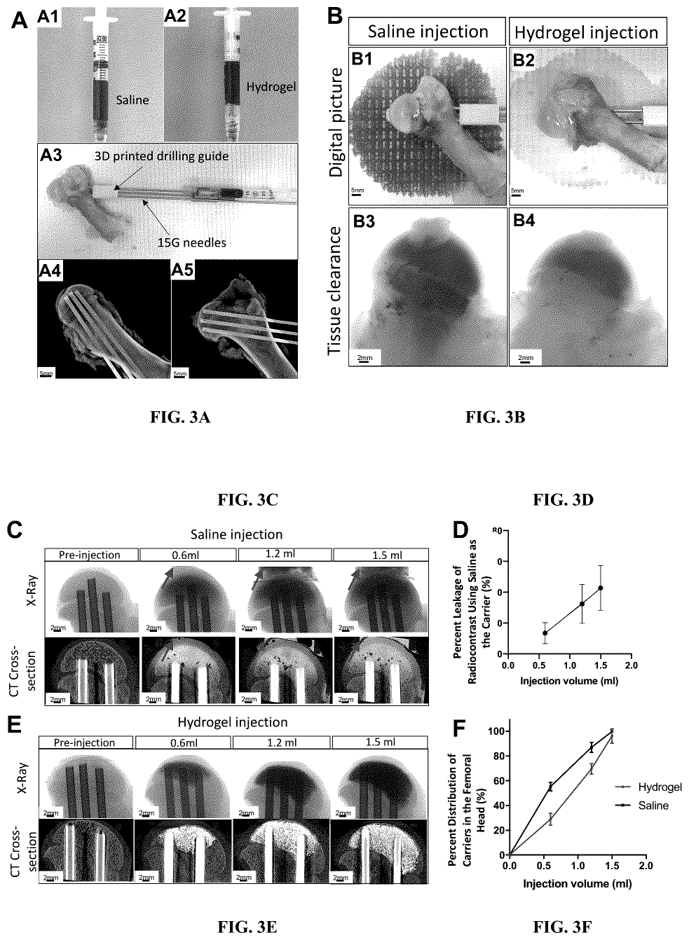

In order to test the bioactivity of the released BMP2 (BMP2-R), osteogenic assays were performed using pig bone marrow mesenchymal cells (pBMMC). The cells were cultured for five days. The qPCR data (FIG. 2C) showed that the pBMSCs exhibited an upregulation in gene expression for Collagen type 1 (Col 1), alkaline phosphate (ALP) and Osteorix (OSX) in the BMP2-R group compared to the cells cultured in osteogenic medium (OM, p<0.0001) and growth medium (GM, p<0.0001). The representative images of Alizarin red staining showed that the pBMMCs produced increased minerals after eleven days of culture in the BMP2-R group than the OM group (FIG. 2D). No significant differences were observed in the osteogenesis of pBMMC between BMP2-R and BMP2 groups (FIGS. 3 C&D). These data indicate that BMP2 released from the GHT hydrogel maintained its bioactivity.

Ex Vitro injection of GHT hydrogel. The leakage and distribution of carriers was tested ex vivo using cadaveric femoral heads. The experimental apparatus setup is illustrated in FIG. 3A. The saline and hydrogel were both loaded with blue dye (FIGS. 3A1 & A2) or a radiocontrast agent to visualize the leakage optically or by X-ray. A three-dimensional (3D) printed drilling guide was used for standardized drilling (FIG. 3A3). Before the injection, three intraosseous needles were placed and confirmed by the X-ray (FIG. 3A4 & A5). After a bone wash procedure, 1.5 ml of blue dye-loaded saline was injected. FIG. 3B1 displayed that an abundant amount of blue dye leaked out and stained the paper. In contrast, only residual saline leaked out after the hydrogel injection (colorless wetting on the paper), and no blue dye leakage was observed (FIG. 3B2). The specimens were further processed with gradient alcohol for dehydration, followed by incubation in a refractive index matching solution consisting of benzyl benzoate and benzyl alcohol (BB/BA) for tissue clearance, which allowed for the visualization of blue dye inside the bone. Visual inspection following tissue clearance revealed that the blue dye was present in the metaphysis after saline injection and removal of the intraosseous needles, indicating a backflow of saline through the drill tunnels (FIG. 3 B3). However, the blue dye was not present in the metaphysis tissue after hydrogel injection and removal of the needles, indicating the retention of the hydrogel in the epiphysis (FIG. 3 B4).

The leakage and distribution of the two carriers were further quantified by μCT (FIG. 3 C). Saline injections revealed leakage after the initial 0.6 ml injection, which increased with higher injection volumes. The leaked fluid was collected and the concentration of the radiocontrast was measured by X-ray intensities. The quantitation showed that over 40% of the radiocontrast leaked out of the femoral head when injecting saline. (FIG. 3D). In comparison, no radiocontrast was detected in the leaked fluid after hydrogel injection (FIG. 3E). Both the saline and hydrogel injections showed a wide distribution within the femoral head (FIG. 3F). The saline injection produced a wider distribution at a relatively low injection volume compared to the hydrogel injection. The results of these experiments suggest that using saline as the carrier can result in severe leakage of the loaded makers, but hydrogel is capable of retaining them within the femoral head.

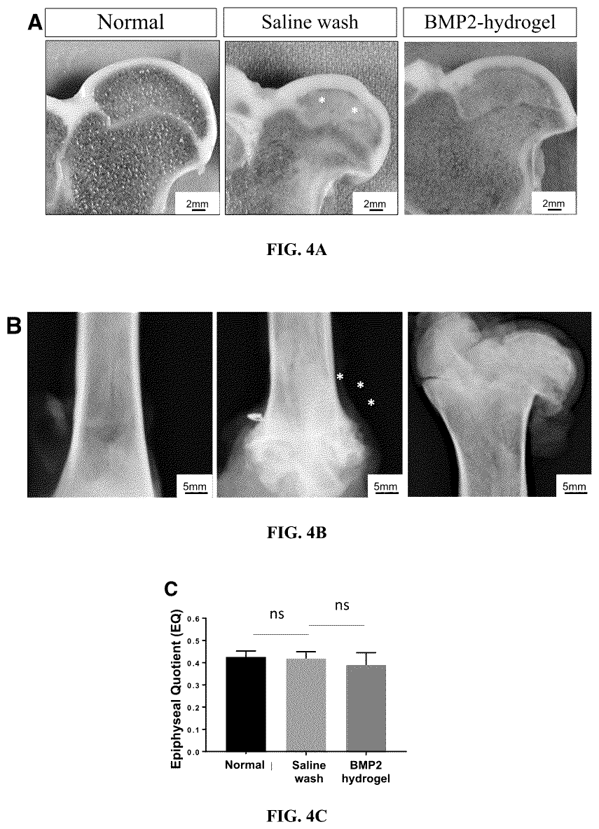

Visual and radiographic assessments of BMP2-hydrogel treatment on a piglet model of LCPD. To investigate the treatment effects in vivo, the BMP2-hydrogel was locally administrated following a bone wash using a minimally invasive percutaneous technique on a piglet model of LCPD, as shown in FIG. 1. After an eight-week period following osteonecrosis induction, the piglets were euthanized, and the femoral heads were retrieved and bisected. As shown in FIG. 4A, the normal femoral head bone marrow appeared brownish, whereas a large region of the bone marrow in the saline wash group appeared whitish. The femoral head bone marrow of the BMP2-hydrogel group appeared brownish in the subchondral region and yellowish in the central region of the epiphysis. On X-ray, bone voids and a discontinued subchondral bone were present in the epiphysis of the saline wash group (FIG. 4B), whereas the epiphysis in the BMP2-hydrogel group had an intact subchondral bone similar to the control group and no bone voids. The deformity of the femoral head was used to evaluate the measurement of the epiphyseal quotient (EQ, maximum femoral head height/maximum femoral head width). The mean value of EQ revealed no significant difference among the three groups (p=0.26, ANOVA) (FIG. 4C).

The hip joint capsule and soft tissues were dissected to evaluate the occurrence of HO. Based on visual inspection and X-ray imaging, HO was not observed in the animals that received the BMP2-hydrogel treatment. The inventors' previous study found ectopic bones in the hip capsule and soft tissue of animals that received the BMP2 saline treatment²¹.

Taken together, visual and radiographic assessments indicate that the BMP2-hydrogel treatment produced a semi-spherical femoral head with a uniform bone matrix, while completely avoiding heterotopic bone formation.

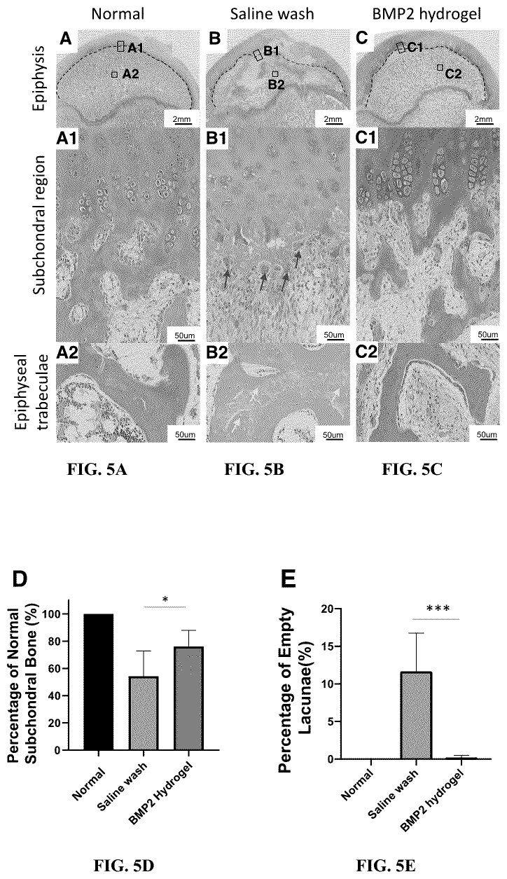

Histological assessments of bone regeneration and remodeling after BMP2-hydrogel treatment. Histological changes of the necrotic femoral head following the different treatments were visualized using H&E staining. Endochondral ossification (EO) was active in the subchondral region of the femoral head in the normal group, with visible hypertrophic chondrocytes columns and active endochondral bone formation (FIG. 5A, black dash line & FIG. 5A1). ONFH halted EO at the subchondral region (FIG. 5B red dash line & FIG. 5B1), with active osteoclastic resorption of the necrotic trabeculae (FIG. 5B1 red arrows) and partial restoration of EO in the subchondral region over time (FIG. 5B, black dash line). In contrast, the BMP2-hydrogel treatment restored most of the subchondral EO, as shown in FIG. 5C1. 76±11% of the subchondral region showed restoration of EO in the BMP2-hydrogel group, compared with 54±17% in the saline wash group (p=0.02, FIG. 5D). The appearance of abundant empty lacunae is the hallmark of osteonecrosis³³. In the saline wash group, a considerable number of empty lacunae were present (yellow arrows) in the epiphyseal trabeculae. However, a significantly decreased number of empty lacunae were found in the BMP2-hydrogel treatment group (FIG. 5E); 11.7±4.5% of osteocyte lacunae were empty in the saline wash group versus 0.2±0.2% in the BMP2-hydrogel group (p=0.0003).

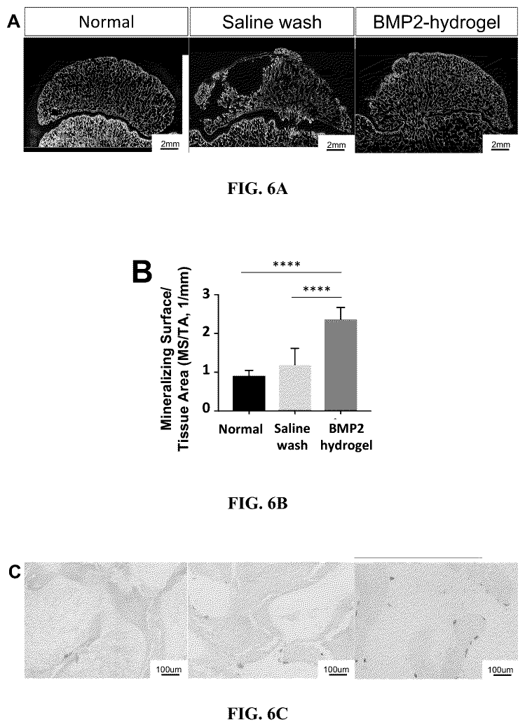

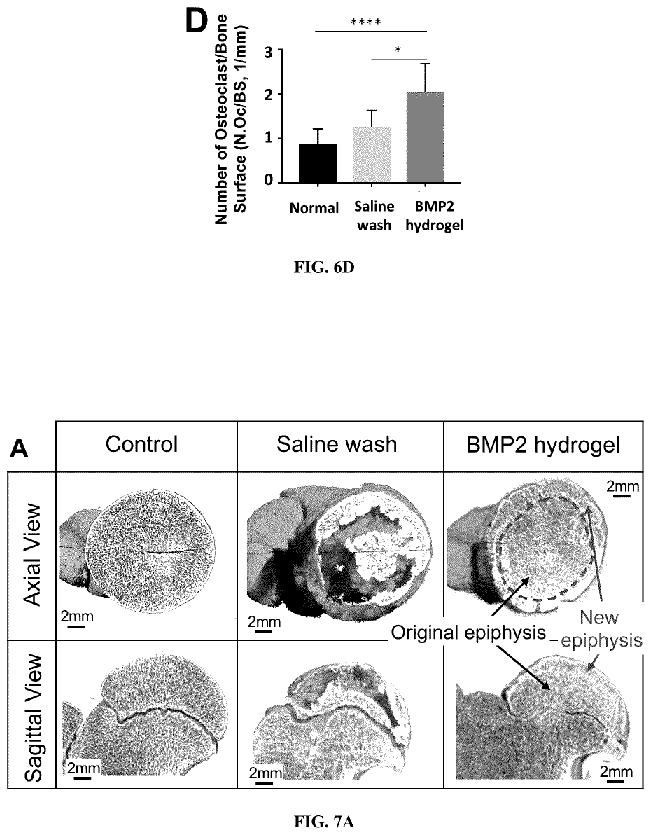

Undecalcified sections with calcine (green) labeling were used to evaluate the mineralizing surface (FIG. 6A). The mineralizing surface per total tissue area (MS/TA) of the BMP2-hydrogel group was significantly increased compared to the saline wash group (2.4±0.3 /mm vs. 1.2±0.4/mm, p<0.0001) and the normal group (0.90±0.1 /mm, p<0.0001) (FIG. 6B). TRAP-stained sections were used to visualize osteoclasts on the epiphyseal trabeculae (FIG. 6C). The number of osteoclasts was significantly higher in the BMP2-hydrogel group compared to the saline wash group (2.2±0.6/mm, vs. 1.6±0.3/mm, p=0.0486) and the normal group (0.7±0.1/mm, p<0.0001). However, the osteoclast number in the BMP2-hydrogel and saline wash groups were significantly higher than the normal group (FIG. 6D). The histological assessments indicate that BMP2-hydrogel accelerated epiphyseal bone regeneration and remodeling following ONFH. It largely restored EO at the subchondral regions, increased trabecular bone formation, and increased bone remodeling as noted by decreased empty lacunae and increased osteoclast number. As a result, most of the necrotic bone in the BMP2-hydrogel group was replaced by new bone.

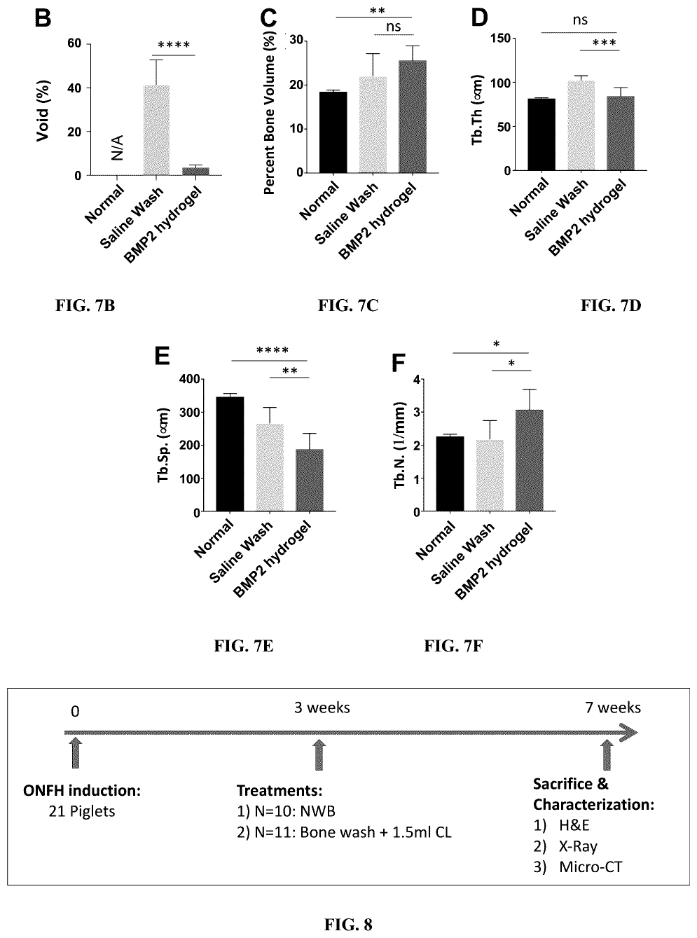

Assessing the epiphyseal bone architecture parameters of the BMP2-hydrogel treatment. μCT was used to evaluate the parameters of the epiphyseal architecture of the femoral head after treatment. Typical 3D images from the three groups are shown in FIG. 7A. Both axial and sagittal views revealed a homogenous presence of trabecular bone in the normal and BMP2-hydrogel treated femoral heads. In the BMP2-hydrogel group, a visible addition of new bone (red arrow) was observed around the original epiphysis (black arrow), which confirms the restoration of EO in the subchondral region. In contrast, the femoral head of the saline wash group showed discontinuous new bone in the subchondral region with large bone voids throughout the epiphysis.

Histomorphometric measurements showed that 41% of the epiphyseal bone void was detected in the saline wash group, which is greater by eleven-fold than the BMP2-hydrogel treated group (3%, p<0.0001) (FIG. 7B). The bone volume per tissue volume (BV/TV) of the BMP2-hydrogel group (25.6±0.3%) was similar to the saline wash group (22.1±4.6%, p=0.22), but it was significantly higher than the normal group (18.5±0.4%, p=0.0085) (FIG. 7C). Mean trabecular thickness (Tb.Th.) was significantly decreased in the BMP2-hydrogel group compared to the saline wash group (84.3±9.9 vs. 102.5±4.9 μm, p=0.0005), but no significant difference was found between the BMP2-hydrogel and normal groups (81.7±0.9 μm, p=0.77, FIG. 7D). Mean trabecular separation (Tb.Sp.) was significantly decreased in the BMP2-hydrogel group (188±48 μm) compared to the saline wash group (267±47 μm, P=0.0084) and the normal group (347 ±10 μm, p<0.0001) (FIG. 7E). Mean trabecular number (Tb.N.) was significantly increased in the BMP2-hydrogel group (3.1±0.6/mm) compared to the saline wash group (2.2±0.6 /mm, p=0.015) and the normal group (2.3±0.6/mm, p=0.026). (FIG. 7F). Taken together, this data suggest that the BMP2-hydrogel treatment achieved homogeneous bone regeneration.

LCPD is a severe pediatric bone disease that can lead to disabling osteoarthritis. Local delivery of biological agents offers potential new treatment options²⁰. However, there is a lack of effective delivery methods which would provide a broad and sufficient local osteoinduction for homogenous bone regeneration. To address this clinical need, the inventors developed a BMP2-hydrogel treatment via a transphyseal bone wash and subsequential injection of BMP2-loaded hydrogel. The inventors found that the new BMP2 delivery strategy can provide broad BMP2 distribution within the necrotic head with no leakage during the injection, thereby restricting the loaded BMP2 within the target region for local osteogenic induction. The GHT hydrogel can retain the bioactive BMP2 for four weeks in vitro. The in vivo experiments using a piglet model of LCPD showed that the BMP2-hydrogel treatment significantly increased the restoration of endochondral ossification at the subchondral region, and produced a near-complete healing of the epiphyseal bone while preventing HO.

Bone wash reconditions the local necrotic microenvironment and facilitates the distribution of biomaterial. The harsh necrotic microenvironment is one of the major challenges for bone repair following ONFH. ONFH produces and leaves an abundance of necrotic debris and pro-inflammatory factors in the bone marrow space, including necrotic fat and debris as well as damage-associated molecular patterns. The necrotic debris activates and sustains local innate immune responses, leading to chronic inflammation, increased bone resorption, and decreased bone formation^{34,35}. To improve the local microenvironment, traditional core decompression procedures are used to remove a large piece of necrotic bone (8-10 mm). However, the procedure raises concerns for iatrogenic complications such as subtrochanteric fracture, inadvertent penetration, or collapse of the femoral head³⁶. Here, the inventors applied three epiphyseal drillings and followed with an intraosseous bone wash, which minimized the disruption of the native trabecular network by using small drillings (≤3 mm). It has been reported that multiple epiphyseal drillings (MED) could produce multiple dispersive tunnels to the necrotic bone, which may be more effective for vascular restoration than one large tunnel¹¹. More importantly, MED drilling tunnels can be used as inflow and outflow portals for the intraosseous saline wash. The inventors' previous study reported that the bone wash following the MED can significantly remove debris in the necrotic bone marrow space such as fats, DNA fragments, and pro-inflammatory proteins^{29,37}. As a result, the washed epiphyseal bone provides a “porous scaffold” facilitating new tissue ingrowth and angiogenesis. The study also revealed that the bone wash process could significantly improve bone regeneration following ONFH, as compared with the MED drillings or with no treatment on the piglet LCPD model³⁷. A new analysis of the bone wash group also presented in this study, FIGS. 4-6. Yet, MED and bone wash procedures could not produce complete regeneration of the necrotic epiphysis, and large bone voids were observed within the femoral head (FIGS. 4 to 6). Moreover, nearly half of the subchondral region exhibited halted endochondral ossification (FIG. 5). With incomplete healing and restoration of endochondral ossification, there is a high risk of development and progression of femoral head deformity37.

GHT hydrogel provides an ideal carrier for local BMP2 administration in the ONFH treatment. Local delivery of BMP2 can dramatically improve osteoinduction in the treatment of ONFH. However, the major concerns of BMP2 are HO and long-term bioactivities. A high dose of BMP2 is commonly needed for long-lasting osteoinduction, which further increases the risk of HO. A prospective clinical study reported a more frequent occurrence of HO in patients receiving BMP2 (4 mg per hip, 8/66 hips) than those not receiving BMP2 (1/75 hips)³⁸. In the inventors' previous study, local BMP2 administration (1 mg BMP2 per hip) exhibited a high incidence of HO when using saline as a carrier (4/6 hips)²¹. The current study also showed over 40% saline leakage could be found in the ex vivo model. These indicates the leakage of the BMP2 contributing the HO formation.

The GHT hydrogel system provides three features for the successful BMP2 delivery, leading to a promising clinical application. Firstly, the preparation of the BMP2 hydrogel is simple. Four solutions of GHT precursor, BMP2, HRP, and H₂O₂ can be aliquoted to a predetermined concentrations and volumes and stored at −80° C. When needed, the solutions can be mixed in regular syringe under room temperature. The preparation of BMP2—hydrogel can be completed within 2 mins by gentle shaking and standing.³²

Secondly, the hydrogel system is friendly to proteins, and sustains the release of BMP2 for four weeks. The crosslinking of the hydrogel involved in the use of HRP and H₂O₂. Although no previous study reported the negative impacts of HRP on BMP2, a high dose of H₂O₂ can inactivate bioactive proteins, including BMP2, due to its strong oxidizing feature.⁴⁸ On the other hand, H₂O₂ in a low concentration is an important mediator of intracellular processes.⁴⁹ The physiological concentration of H₂O₂ in plasma ranges from 1 μM to 5 μM.⁵⁰ It indicates that a low residual level of H₂O₂ would not be detrimental to bioactive proteins. In the current system, the final concentrations of HRP and H₂O₂ were 1 unit/ml and 1 mol/ml, respectively. Given the catalytic effect of HRP on the dissociation of H₂O₂, a low residual level of H₂O₂ can be anticipated in the hydrogel after 2 mins of hydrogel gelation. Moreover, heparin could specifically bind to the BMP2 and prevent it from denaturation and degradation.^{51,52} Therefore, a high BMP2 bioactivity was obtained, and the in vitro data showed that over 80% of the bioactive BMP2 was detected (FIG. 2).

Thirdly, the physical features of the GHT hydrogel play an important role in maintaining a high local osteoinduction and preventing HO formation. The injectability of the GHT hydrogel system can be easily adjusted by changing the precursor concentration³². Compared to a saline carrier, injection of 2% GHT hydrogel required higher injection pressure due to its lower flowability. The feature, on the other side, produced a smooth hand-injection, and ensured the hydrogel stay at the trabecular space without leakage (FIG. 3). However, when injecting the hydrogel, a conservative injection volume is recommended to avoid leakage by overdose. The injection volume should be determined according to the size of the femoral head. For an eight to nine weeks old male Yorkshire piglet, the total marrow space of the femoral head ranged from 1.8 ml-2 ml. Therefore, 1.5 ml of the hydrogel was applied for both in vivo and ex vivo experiments, and no leakage was observed.

As a result, the BMP2-hydrogel treatment produced robust osteoinduction within the necrotic head. After seven weeks of treatment, the BMP2-hydrogel treated femoral heads exhibited homogenous bone regeneration (FIG. 6). The epiphyseal bone showed a high level of bone formation and remodeling, which was reflected by a low ratio of empty lacunae (FIG. 6E) and increased bone formation (MS/TA, FIG. 7B) and bone resorption (N.Oc/TA, FIG. 7D). The inventors also observed a high ratio of restored endochondral ossification in the subchondral region (FIG. 6D). Compared to the previous study which used saline for BMP2 delivery, the inventors found less femoral head deformity, more homogeneous bone regeneration, and avoided HO in the current study²¹. Therefore, the use of GHT hydrogel for the local delivery of BMP2 provided a broad and effective bone regeneration for ONFH.

In summary, local BMP2-hydrogel injections after a bone wash procedure using a multi-needle technique produced homogeneous bone regeneration while preventing HO. The combined treatment of the BMP2-hydrogel and the bone wash technique may be a potential ONFH treatment for teenagers and young adults with inactive growth plates.

Materials. Gelatin (Type B, from bovine skin, 225 g Bloom, average molecular weight=50 kDa, Cat #G9391), heparin (sodium salt from porcine intestinal mucosa, MW=17-19 kDa), Type I collagenase (from clostridium histolyticum), calcein (Ex/Em 495/517 nm, cat. no. 00875) were purchased from Sigma Aldrich (St Louis, MO, USA). The gelatin-tyramine-heparin hydrogel precursor (GTH) was synthesized as previously reported³². Horseradish peroxidase (HRP, 304 units/mg) was purchased from Thermo Scientific (Rockford, IL, USA). Hydrogen peroxide (H₂O₂) aqueous solution (35%, w/w) was purchased from BDH Chemicals (Westchester, PA, USA). The radio-contrasts, iopamidol (Isovue) and barium sulfate (BaSO₄) suspension were purchased from Bracco (Milan, Italy). The recombinant human BMP2 powder was obtained from INFUSE® Bone Graft (Medtronic, Minneapolis, MN) and reconstituted in sterile water according to instructions from manufacture. BMP2 Quantikine ELISA Kits were purchased from R&D Systems, Inc. (Minneapolis, MN, USA).

Preparation of GTH hydrogel and BMP2-hydrogel. The solution of GTH hydrogel precursors was prepared by dissolving GTH hydrogel precursors into phosphate-buffered saline (PBS, Gibco). For the ex vivo study, GTH hydrogel was prepared by mixing 1.5 ml of the GTH solution (2%, w/v), 7.5 ul of HRP solution (200 unit/ml), 7.5 ul of peroxide hydrogen (0.2M), and one drop of blue dye (Salis International, Inc., Oceanside, CA) in a 3 ml syringe. The mixture was gently shaken by hand, and then left standing at room temperature for 2 mins.

For in vivo studies and release kinetics, all the solutions were sterilized by passing through the 0.22 μm filter (Corning™ Disposable Vacuum Filter). 1.5 ml of BMP2-hydrogel was prepared by mixing 0.75 ml of BMP2 solution (1.34 mg/ml), 0.75 ml of GTH solution (4%, w/v), 7.5 μl of HRP solution (200 unit/ml) and 7.5 μl of peroxide hydrogen (0.2 M) in a 3 ml syringe with cap at room temperature. After 2 mins setting, the liquid mixture turned to hydrogel due to the chemical crosslinking. It was kept at 4° C. for short term storage before injection. At the time of injection, the hydrogel was warmed to 37° C. and it was in a gel state. The concentration of BMP-2 chosen for this study was based on the inventors' previous results²¹.

In Vitro Release BMP-2 from the GTH Hydrogel. 100 ul of BMP2-hydrogel was injected through 24 G needles into the insert of a 24-well transwell system. The insert and the BMP2-hydrogel were incubated in 1 ml PBS buffer at 37° C. At a specific time point, the release medium was collected and stored at −80 ° C. with 1 ml fresh medium replaced. At the last collection, the gels were soaked in 1 ml release buffer containing 30 units/ml type I collagenase, and the BMP-2 in the final solution was defined as unreleased BMP-2. After the dissolution of the gel, all the samples were thawed and quantified using a BMP-2 Quantikine ELISA Kit (R&D systems). The initially bound BMP-2 was determined by adding all the released BMP-2 and unreleased BMP-2 together. Three samples were prepared at each time point, and each experiment was repeated twice.

Ex Vivo Injection of GTH Hydrogel. The specimens were collected under a clean condition in an operating room, wrapped in saline-soaked gauze, and stored at −20° C. To mimic the epiphyseal osteonecrosis ex vivo, the samples were subjected to three thaw-freeze cycles. For each cycle, the samples were placed into a 37° C. water bath for 6 hours, followed by a −20° C. freezer for over 4 hours. After that, three 15-gauge intraosseous needles were placed within the femoral epiphysis using a transphyseal approach and a 3D-printed needle guide for parallel needle placement. The inter-needle distance was 8 mm. The needle placement was considered satisfactory based on X-ray imaging when the needles were in the mid-coronal plane of the epiphysis, crossing the physis by at least 2.5 mm. Then, an epiphyseal bone wash was performed to remove bone marrow debris. Two 30 mL syringes were connected to two of three needles using a Luer-Lock. The three intraosseous needles were used alternatively as inflow and outflow portals for the saline wash. 30 mL of pre-warmed saline was used, and 16 washes were performed per sample with a total wash volume equaling 480 mL per sample.

For characterization of hydrogel distribution, 1.5 ml of the hydrogel was manually injected into the epiphysis with 0.5 ml for each needle. To visualize the leakage, the inventors loaded blue dye or radiocontrast during the preparation of the hydrogel (n=6). The injection of saline (with blue dye or radiocontrast) was used as the control (n=6).

Animals. The study was approved by the local Institutional Animal Care and Use Committee at the University of Texas Southwestern Medical Center (Protocol number: 2016-101442-USDA). A total of twelve male Yorkshire piglets aged between six to eight weeks (25 to 35 lbs.) were obtained from a breeder for the in vivo study (K-Bar Livestock, LLC, Sabinal, TX). All animals were maintained under environmental controls consistent with the Guide for the Care and Use of Laboratory Animals.

Induction of Osteonecrosis and Local Administration of BMP2-Hydrogel. Ischemic osteonecrosis in the femoral head of a piglet was induced on the right limb as previously described³⁷. One week following the induction, the piglets received three percutaneous epiphyseal drillings using three 15-gauge intraosseous needles, followed by the bone wash procedure using 480 ml prewarmed saline. After the bone wash, 1.5 ml of BMP2-hydrogel was injected through the three drilling needles (0.5 ml per needle). All piglets received a local non-weight-bearing treatment via above-knee amputation using a previously described method on the right limb³⁷. The non-weight-bearing treatment was instituted postoperatively to minimize femoral head deformity and to simulate the clinical practice of recommending local non-weight-bearing treatment postoperatively in patients with active stage of LCPD.

μCT and Morphometric Assessment. Following euthanasia, the femoral heads were bisected coronally and fixed in 10% neutral buffered formalin. After fixation, all femoral heads were scanned using a μCT (Skyscan 1172, Bruker-μCT, Kontich, Belgium) at a setting of 100 kV and 100 μA and a resolution of 13.3 μm/pixel as previously described³⁷ and reconstructed with NRecon (version 1.7.0.4; Bruker-μCT, Kontich, Belgium). The reconstructed images were binarized to a common threshold using CTAn (version 1.13.5.1; Bruker μCT), where the region of interest was defined to capture the original necrotic epiphysis. The region of interest was outlined within the epiphysis, avoiding the subchondral region of the calcified epiphyseal cartilage. The CTAn software was used to calculate the three-dimensional morphometric values for percent bone volume, trabecular thickness, trabecular number, trabecular separation, and percent bone void volume in the epiphysis of the femoral heads. To determine a bone void volume, spaces with a trabecular separation larger than 570 μm were defined as bone voids, as the control group had a maximum trabecular separation of 570 μm.

μCT Assessment of Epiphyseal Quotient. Anteroposterior X-ray images were used to measure the epiphyseal quotient that reflects the amount of femoral head collapse. The epiphyseal quotient is defined as the maximum height divided by the maximum width of the femoral head⁴⁵.

Histology, bone histomorphometry, and fluorochrome labeling Analysis. The bisected femoral head specimens were dehydrated in a series of graded ethanol solutions after μCT scanning. The anterior half of the femoral head was processed in methyl methacrylate (MMA) for plastic embedding, whereas the posterior half of the femoral head was decalcified in ethylenediaminetetraacetic acid (EDTA) and embedded in paraffin. Both portions were sectioned at a thickness of 4 μm.

The paraffin sections were used to perform hematoxylin and eosin (H&E) staining using a standard protocol⁴⁶. The percentage of the restored subchondral endochondral ossification was evaluated using H&E stained images by measuring the length of the osteochondral junction with new bone formation, normalizing the value with the total length of the osteochondral junction. The empty osteocyte lacunae were counted, as defined by osteocyte lacunae with an absence of the cell body or lacunae containing only a pyknotic nucleus⁴⁷.

The plastic sections were stained with tartrate-resistant acid phosphatase (TRAP) to determine the number of osteoclasts³⁷. TRAP-positive cells were counted and normalized to bone surface (N/BS). Plastic sections were also imaged to determine the percentage of mineralizing surface via quantifying the fluorochrome-labeled bone surface per tissue area (MS/TA). All sections were imaged using the OSTEOIMAGER Scanner and analyzed using the BIOQUANT OSTEO software.

Statistical Analysis. For the comparison of injection pressure, a student t-test was performed to determine the difference between 2 groups. For the epiphyseal quotient, μCT morphometric, histologic, and histomorphometric measurements, a one-way analysis of variance (ANOVA) was performed to determine the overall difference among the 3 groups. If the difference was significant (p<0.05), a post-hoc Tukey honestly significant difference test was performed to assess the significance among groups.

In vivo results of clodrosome treatment in the piglet model of LCPD.

As FIG. 8 is a flowchart that illustrates the testing model using twenty-one piglets (15 male and 6 female) were induced with ischemic osteonecrosis in the femoral head (ONFH) on the right side. Three weeks following the induction, the piglets were randomly assigned to the non-weight-bearing (N=10) or the Clodrosome (CL) treatment (N=11) groups. For the CL treatment, the piglets received three percutaneous epiphyseal drillings using three 15-gauge intraosseous needles, followed by the bone wash procedure. Then, 1.5 ml of CL was injected through the three drilling needles (0.5 ml per needle). All piglets received a local non-weight-bearing (NWB) treatment via above-knee amputation of the right limb.

Three weeks following the treatments, the animals were sacrificed and imaged by X-ray. Then, the femoral heads were fixed in 10% neutral buffered formalin. After fixation, all femoral heads were scanned by micro-CT (mCT). Next, the femoral head specimens were processed for paraffin embedding. The paraffin sections were used to perform hematoxylin and eosin (H&E) staining.

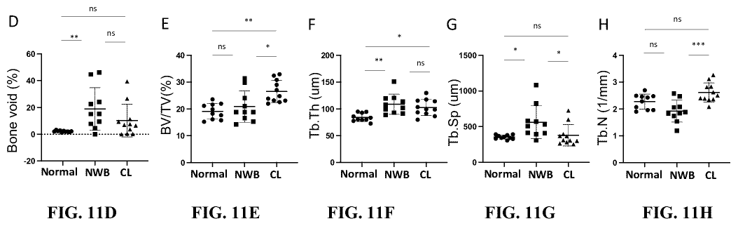

Histological assessments of bone repair after CL treatment. Histological changes of the necrotic femoral head following the different treatments were visualized using H&E staining. Endochondral ossification (EO) was active in the subchondral region of the femoral head in the normal group, with visible columns of hypertrophic chondrocytes and active endochondral bone formation (FIG. 9A, black dash line and FIG. 9A1). Most of the EO was halted due to the osteonecrosis at the subchondral region in the NWB group (FIG. 9B red dash line AND FIG. 9B1). In contrast, a larger area of restoration of EO was observed in some areas of the subchondral region in the CL group (FIG. 9C black dash line ND FIG. 9C1). As shown in FIG. 9D, 39±15% of the subchondral region showed restoration of EO in the CL group, compared with 21±8% in the NWB group (p=0.04). Moreover, active osteoclastic bone resorption was observed in the epiphysis of the NWB group, resulting in large areas of trabeculae bone loss (FIG. 9B, FIG. 11. 9B2 red arrows). In contrast, active appositional bone formation was observed in some areas of the epiphysis in the CL group (FIG. 9C2).

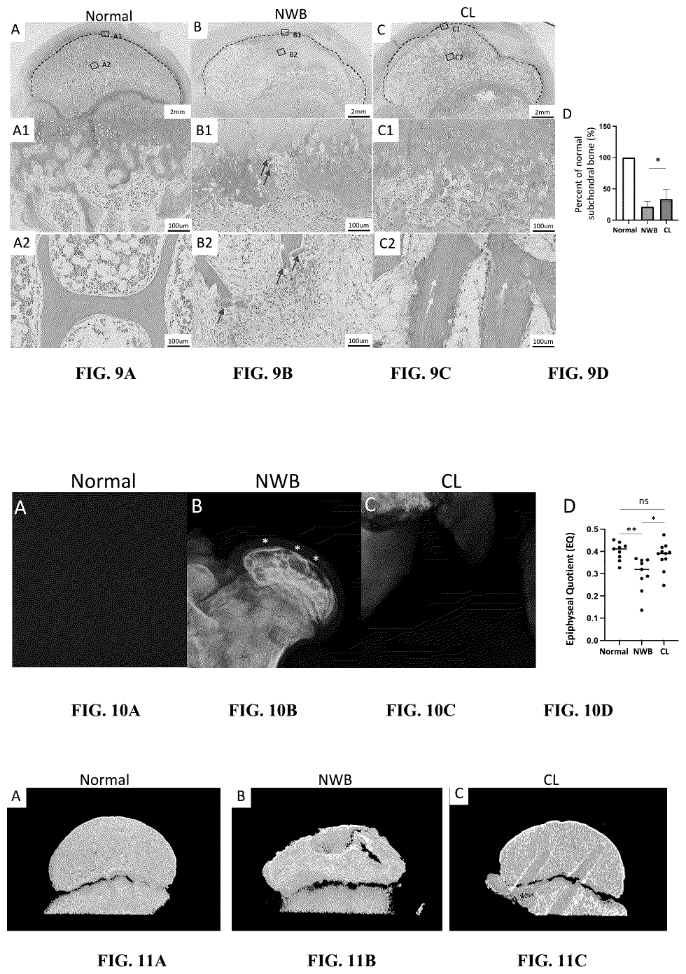

Radiographic assessments of bone repair after CL treatment. X-ray imaging of the normal femoral heads showed a semi-spherical shape with homogeneous radiodensity in the epiphysis. In the NWB group, bone voids and subchondral bone disruption were present in the epiphysis. The CL-treated group showed a semi-spherical shape with fewer areas of bone voids. The EQ value revealed no significant difference between the normal and CL-treated femoral heads (0.40±0.04 vs. 0.38±0.06, p=0.74). The EQ value of the CL-treated group was significantly higher than the NWB group (0.38±0.06 vs. 0.30±0.08, p=0.016) (FIG. 10D)

mCT was used to qualitatively and quantitively evaluate the epiphyseal architecture of the femoral head after treatment. Typical 3D reconstructed images of sagittal views from the three groups are shown in FIGS. 11A-11C. An even distribution of trabecular bone matrix was observed in the epiphysis of the normal group (FIG. 11A). In contrast, large bone voids were present in the epiphysis of the NWB group. (FIG. 11B). In the CL group, the trabecular bone matrix was more homogeneous like the normal group (FIG. 11C).

Histomorphometric measurements showed that 19% of the tissue volume in the epiphysis was occupied by bone voids in the NWB group compared to 10% in the CL group (p<0.01). (FIG. 11D). The trabecular bone volume/tissue volume (BV/TV) of the CL group (26.5±4.1%) was significantly higher than the normal (19.1±2.3%, p=0.002), and the NWB (20.9±5.8%, p=0.02) groups (FIG. 11E). The trabecular thickness (Tb.Th.) was significantly increased in the CL group compared to the normal group (100.3±15.6 mm vs. 84.3±2.6 μm, p=0.02), but no significant difference was found between the CL and the NWB (100.3±15.6 mm vs. 108.8±18.8 μm, p=0.65) groups (FIG. 11F). The trabecular separation (Tb.Sp.) of the CL group was significantly decreased compared to the NWB group (379.2±157.7 μm vs. 562.2±230.9 μm, p=0.2, FIG. 11G). It did not show a significant difference compared to the normal group (379.2±157.7 μm vs. 354.4±29.3 μm, p=0.93). The trabecular number (Tb.N.) of the CL group was significantly increased compared to the NWB group (2.61±0.37 μm vs. 1.92±0.4 μm, p=0.0003, FIG. 11H). It did not show a significant difference compared to the normal group (2.61±0.37 μm vs. 2.27±0.28 μm, p=0.09).

In summary, CL treatment following osteonecrosis preserved the semi-spherical shape of the femoral head by protecting the epiphyseal trabeculae from bone resorption, increasing new bone formation, and improving the recovery of the endochondral ossification in the subchondral region.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1 Molloy, M. K. & MacMahon, B. Incidence of Legg-Perthes disease (osteochondritis deformans). N Engl J Med 275, 988-990, doi:10.1056/NEJM196611032751804 (1966).
2 Stulberg, S. D., Cooperman, D. R. & Wallensten, R. The natural history of Legg-Calve-Perthes disease. J Bone Joint Surg Am 63, 1095-1108 (1981).
3 Joseph, B., Mulpuri, K. & Varghese, G. Perthes' disease in the adolescent. The Journal of bone and joint surgery. British volume 83, 715-720, doi:10.1302/0301-620x.83b5.10663 (2001).
4 McAndrew, M. P. & Weinstein, S. L. A long-term follow-up of Legg-Calvé-Perthes disease. J Bone Joint Surg Am 66, 860-869, doi:10.2106/00004623-198466060-00006 (1984).
5 Zhao, D. et al. Guidelines for clinical diagnosis and treatment of osteonecrosis of the femoral head in adults (2019 version). J Orthop Translat 21, 100-110, doi:10.1016/j.jot.2019.12.004 (2020).
6 Kim, H. K. Pathophysiology and new strategies for the treatment of Legg-Calvé-Perthes disease. J Bone Joint Surg Am 94, 659-669, doi:10.2106/jbjs.J.01834 (2012).
7 Weinstein, S. L. Bristol-Myers Squibb/Zimmer award for distinguished achievement in orthopaedic research. Long-term follow-up of pediatric orthopaedic conditions. Natural history and outcomes of treatment. J Bone Joint Surg Am 82-a, 980-990, doi:10.2106/00004623-200007000-00010 (2000).
8 Herring, J. A., Kim, H. T. & Browne, R. Legg-Calve-Perthes disease. Part II: Prospective multicenter study of the effect of treatment on outcome. J Bone Joint Surg Am 86, 2121-2134 (2004).
9 Aruwajoye, O. O., Monte, F., Kim, A. & Kim, H. K. W. A Comparison of Transphyseal Neck-Head Tunneling and Multiple Epiphyseal Drilling on Femoral Head Healing Following Ischemic Osteonecrosis: An Experimental Investigation in Immature Pigs. J Pediatr Orthop, doi:10.1097/BPO.0000000000001219 (2018).
10 Song, W. S., Yoo, J. J., Kim, Y. M. & Kim, H. J. Results of multiple drilling compared with those of conventional methods of core decompression. Clin Orthop Relat Res 454, 139-146, doi:10.1097/01.blo.0000229342.96103.73 (2007).
11 Al Omran, A. Multiple drilling compared with standard core decompression for avascular necrosis of the femoral head in sickle cell disease patients. Arch Orthop Trauma Surg 133, 609-613, doi:10.1007/s00402-013-1714-9 (2013).
12 Bozic, K. J., Zurakowski, D. & Thornhill, T. S. Survivorship Analysis of Hips Treated with Core Decompression for Nontraumatic Osteonecrosis of the Femoral Head*. JBJS 81 (1999).
13 Chughtai, M. et al. An evidence-based guide to the treatment of osteonecrosis of the femoral head. Bone Joint J 99-b, 1267-1279, doi:10.1302/0301-620x.99b10.Bjj-2017-0233.R2 (2017).
14 Mont, M. A., Carbone, J. J. & Fairbank, A. C. Core Decompression Versus Nonoperative Management for Osteonecrosis of the Hip. Clinical Orthopaedics and Related Research® 324 (1996).
15 Carragee, E. J., Hurwitz, E. L. & Weiner, B. K. A critical review of recombinant human bone morphogenetic protein-2 trials in spinal surgery: emerging safety concerns and lessons learned. Spine Journal 11, 471-491, doi:10.1016/j.spinee.2011.04.023 (2011).
16 Simmonds, M. C. et al. Safety and Effectiveness of Recombinant Human Bone Morphogenetic Protein-2 for Spinal Fusion A Meta-analysis of Individual-Participant Data. Annals of Internal Medicine 158, 877-+, doi:10.7326/0003-4819-158-12-201306180-00005 (2013).
17 Carreira, A. C. et al. Bone Morphogenetic Proteins Facts, Challenges, and Future Perspectives. Journal of Dental Research 93, 335-345, doi:10.1177/0022034513518561 (2014).
Garrison, K. R. et al. Clinical effectiveness and cost-effectiveness of bone morphogenetic proteins in the non-healing of fractures and spinal fusion: a systematic review. Health Technol Assess 11, 1-150, iii-iv, doi:10.3310/hta11300 (2007).
19 Sun, W. et al. Recombinant human bone morphogenetic protein-2 in debridement and impacted bone graft for the treatment of femoral head osteonecrosis. PLoS One 9, e100424, doi:10.1371/journal.pone.0100424 (2014).
20 Kim, H. K., Aruwajoye, O., Du, J. & Kamiya, N. Local administration of bone morphogenetic protein-2 and bisphosphonate during non-weight-bearing treatment of ischemic osteonecrosis of the femoral head: an experimental investigation in immature pigs. J Bone Joint Surg Am 96, 1515-1524, doi:10.2106/jbjs.M.01361 (2014).
21 Vandermeer, J. S. et al. Local administration of ibandronate and bone morphogenetic protein-2 after ischemic osteonecrosis of the immature femoral head: a combined therapy that stimulates bone formation and decreases femoral head deformity. J Bone Joint Surg Am 93, 905-913, doi:10.2106/jbjs.J.00716 (2011).
22 Chen, N. F. et al. Symptomatic ectopic bone formation after off-label use of recombinant human bone morphogenetic protein-2 in transforaminal lumbar interbody fusion. J Neurosurg Spine 12, 40-46, doi:10.3171/2009.4.Spine0876 (2010).
Deutsch, H. High-dose bone morphogenetic protein-induced ectopic abdomen bone growth. Spine J 10, el-4, doi:10.1016/j.spinee.2009.10.016 (2010).
24 Wong, D. A., Kumar, A., Jatana, S., Ghiselli, G. & Wong, K. Neurologic impairment from ectopic bone in the lumbar canal: a potential complication of off-label PLIF/TLIF use of bone morphogenetic protein-2 (BMP-2). Spine J 8, 1011-1018, doi:10.1016/j.spinee.2007.06.014 (2008).
Lieberman, J. R., Conduah, A. & Urist, M. R. Treatment of osteonecrosis of the femoral head with core decompression and human bone morphogenetic protein. Clin Orthop Relat Res, 139-145, doi:10.1097/01.blo.0000150312.53937.6f (2004).
26 Ohta, H. et al. The effects of heat on the biological activity of recombinant human bone morphogenetic protein-2. J Bone Miner Metab 23, 420-425, doi:10.1007/s00774-005-0623-6 (2005).
27 Liu, M. et al. Injectable hydrogels for cartilage and bone tissue engineering. Bone Res 5, 17014, doi:10.1038/boneres.2017.14 (2017).
28 Phipps, M. C., Monte, F., Mehta, M. & Kim, H. K. Intraosseous Delivery of Bone Morphogenic Protein-2 Using a Self-Assembling Peptide Hydrogel. Biomacromolecules 17, 2329-2336, doi:10.1021/acs.biomac.6b00101 (2016).
29 Alves do Monte, F. et al. Development of a novel minimally invasive technique to washout necrotic bone marrow content from epiphyseal bone: A preliminary cadaveric bone study. Orthop Traumatol Surg Res 106, 709-715, doi:10.1016/j.otsr.2020.01.006 (2020).
30 Ma, C., Jing, Y., Sun, H. & Liu, X. Hierarchical Nanofibrous Microspheres with Controlled Growth Factor Delivery for Bone Regeneration. Adv Healthc Mater 4, 2699-2708, doi:10.1002/adhm.201500531 (2015).
31 Ruppert, R., Hoffmann, E. & Sebald, W. Human bone morphogenetic protein 2 contains a heparin-binding site which modifies its biological activity. European Journal of Biochemistry 237, 295-302, doi:10.1111/j.1432-1033.1996.0295n.x (1996).
32 Li, Z. et al. Injectable gelatin derivative hydrogels with sustained vascular endothelial growth factor release for induced angiogenesis. Acta Biomater 13, 88-100, doi:10.1016/j.actbio.2014.11.002 (2015).
33 Fondi, C. & Franchi, A. Definition of bone necrosis by the pathologist. Clin Cases Miner Bone Metab 4, 21-26 (2007).
34 Andreev, D. et al. Osteocyte necrosis triggers osteoclast-mediated bone loss through macrophage-inducible C-type lectin. J Clin Invest 130, 4811-4830, doi:10.1172/jci134214 (2020).
Cao, J. J. Effects of obesity on bone metabolism. Journal of Orthopaedic Surgery and Research 6, 30, doi:10.1186/1749-799X-6-30 (2011).
36 Beltran, J. et al. Core decompression for avascular necrosis of the femoral head: correlation between long-term results and preoperative MR staging. Radiology 175, 533-536, doi:10.1148/radiology.175.2.2326478 (1990).
37 Kim, H. K. W. et al. Minimally Invasive Necrotic Bone Washing Improves Bone Healing After Femoral Head Ischemic Osteonecrosis: An Experimental Investigation in Immature Pigs. J Bone Joint Surg Am 103, 1193-1202, doi:10.2106/jbjs.20.00578 (2021).
38 Shi, L., Sun, W., Gao, F., Cheng, L. & Li, Z. Heterotopic ossification related to the use of recombinant human BMP-2 in osteonecrosis of femoral head. Medicine (Baltimore) 96, e7413, doi:10.1097/md.0000000000007413 (2017).
39 Schmidt, A., Schumacher, J. T., Reichelt, J., Hecht, H. J. & Bilitewski, U. Mechanistic and molecular investigations on stabilization of horseradish peroxidase C. Anal Chem 74, 3037-3045, doi:10.1021/ac0108111 (2002).
40 Makela, E. A., Vainionpaa, S., Vihtonen, K., Mero, M. & Rokkanen, P. The effect of trauma to the lower femoral epiphyseal plate. An experimental study in rabbits. J Bone Joint Surg Br 70, 187-191, doi:10.1302/0301-620X.70B2.3346285 (1988).
41 Janarv, P. M., Wikstrom, B. & Hirsch, G. The influence of transphyseal drilling and tendon grafting on bone growth: an experimental study in the rabbit. J Pediatr Orthop 18, 149-154 (1998).
42 Garces, G. L., Mugica-Garay, I., Lopez-Gonzalez Coviella, N. & Guerado, E. Growth-plate modifications after drilling. J Pediatr Orthop 14, 225-228, doi:10.1097/01241398-199403000-00018 (1994).
43 Tsao, A. K. et al. Biomechanical and clinical evaluations of a porous tantalum implant for the treatment of early-stage osteonecrosis. J Bone Joint Surg Am 87 Suppl 2, 22-27, doi:10.2106/jbj s.E.00490 (2005).
44 Zhang, Y. et al. A new 3D printed titanium metal trabecular bone reconstruction system for early osteonecrosis of the femoral head. Medicine (Baltimore) 97, e11088, doi:10.1097/md.0000000000011088 (2018).
45 Koob, T. J. et al. Biomechanical properties of bone and cartilage in growing femoral head following ischemic osteonecrosis. J Orthop Res 25, 750-757, doi:10.1002/jor.20350 (2007).
46 Gerwin, N., Bendele, A. M., Glasson, S. & Carlson, C. S. The OARSI histopathology initiative—recommendations for histological assessments of osteoarthritis in the rat. Osteoarthritis Cartilage 18 Suppl 3, S24-34, doi:10.1016/j.joca.2010.05.030 (2010).
47 Weinstein, R. S. Glucocorticoid-induced osteonecrosis. Endocrine 41, 183-190, doi:10.1007/s12020-011-9580-0 (2012).
48 Quan Qing, et al, Effects of hydrogen peroxide on biological characteristics and osteoinductivity of decellularized and demineralized bone matrices, J Biomed Mater Res A. 2019 July; 107(7):1476-1490. doi: 10.1002/jbm.a.36662. Epub 2019 Mar. 7. PMID: 30786151
49 Noemi Di Marzo, et al. The Role of Hydrogen Peroxide in Redox-Dependent Signaling: Homeostatic and Pathological Responses in Mammalian Cells. Cells. 2018 Oct. 4; 7(10):156. doi: 10.3390/ce11s7100156. PMID: 30287799
50 Henry Jay Forman, et al., What is the concentration of hydrogen peroxide in blood and plasma? Arch Biochem Biophys. 2016 Aug. 1; 603:48-53. doi: 10.1016/j.abb.2016.05.005. Epub 2016 May 9. PMID: 27173735
51 Ma, et al., Hierarchical Nanofibrous Microspheres with Controlled Growth Factor Delivery for Bone Regeneration. Adv Healthc Mater. 2015 Dec. 9; 4(17):2699-708. doi: 10.1002/adhm.201500531. Epub 2015 Oct. 13. PMID: 26462137
52 Ruppert, et al., Human bone morphogenetic protein 2 contains a heparin-binding site which modifies its biological activity. Eur J Biochem. 1996 Apr. 1; 237(1):295-302. doi: 10.1111/j.1432-1033.1996.0295n.x. PMID: 8620887

What is claimed is:

1. A method of treating bone osteonecrosis comprising:
preparing a bone growth-promoting hydrogel comprising one or more bone growth-promoting agents in a hydrogel, the hydrogel comprising:
at least one of gelatin tyramine (GT), gelatin-heparin tyramine (GHT), gelatin methacryloyl (GelMa), gelatin acryloyl (GelAC), or collagen, or a hydrogel-forming peptide;
at least one bone growth-promoting agent or a macrophage depletion reagent; and
optionally one or more pharmaceutically acceptable carriers; and
injecting the bone growth-promoting hydrogel into one or more openings in a bone, wherein the bone growth-promoting hydrogel treats bone osteonecrosis.

2. The method of claim 1, wherein the growth-promoting agent or macrophage depletion reagent is selected from bone morphogenic protein 2 (BMP-2), bone morphogenic protein 7 (BMP-7), Vascular endothelial growth factor (VEGF), clodronate, or Clodrosome.

3. The method of claim 1, wherein the composition is a biocompatible isotonic fluid and optionally comprises biocompatible detergents, biocompatible surfactants, biocompatible alcohols, antibiotics, preservatives, or combinations thereof, wherein the washing fluid cleans at least 50, 60, 70, 75, 80, 85, or 90% of the volume within the bone.

4. The method of claim 1, further comprising at least one of autologous bone marrow, autologous bone stem cells, bone graft material, bone void filler, cancellous bone graft or fragments, osteoconductive material, osteoproliferative material, osteoinductive material, or bone material infused collagen matrix.

5. The method of claim 1, wherein the GT is a Gelatin tyramine hydrogel, a gelatin-heparin tyramine (GHT) hydrogel, or both (1 to 10% (w/v)), which comprises a trace amount of hydrogen peroxide (0.1 mM-10 mM), a horseradish peroxidase enzyme (0.1-10 unit/ml), wherein the bone growth promoting agent or a macrophage depletion reagent retains at least 80% bioactivity, and optionally comprising heparin at (0.5-5% w/v) that is formed ex vivo or in situ.

6. The method of claim 1, wherein the GelMa is a Gelatin methacryloyl (GelMa) hydrogel, a gelatin acryloyl (GelAC) hydrogel, or both (2 to 10% (w/v)), which comprises a photoinitiator at (0.01-1% w/v) and the hydrogel is formed by ultraviolet light ex vivo or in situ.

7. The method of claim 1, wherein the collagen is a collagen hydrogel (0.3-3%, w/v) formed by gelation at 37° C. without the addition of crosslinkers that is formed ex vivo or in situ.

8. The method of claim 1, wherein the bone is a femoral head, a humeral head, a knee condyle, a proximal tibia, or an ankle talus.

9. The method of claim 1, wherein the osteonecrosis is Legg-Calvé-Perthes Disease, idiopathic (unknown cause), due to corticosteroid, trauma, alcohol, sickle cell disease, or other known causes of osteonecrosis.

10. The method of claim 1, further comprising the step of washing an interior of the bone prior to injecting the bone growth-promoting hydrogel.

11. A composition for treating bone osteonecrosis comprising:
at least one of a Gelatin tyramine (GT), a gelatin-heparin tyramine (GHT), a gelatin methacryloyl (GelMa), a gelatin acryloyl (GelAC), collagen, or a hydrogel-forming peptide that is formed ex vivo or in situ for the treatment of bone osteonecrosis and that prevent or reduce leakage of an active agent, wherein the active agent comprises at least one bone growth promoting agent or a macrophage depletion reagent in an amount sufficient to reduce or eliminate bone osteonecrosis; and
optionally one or more pharmaceutically acceptable carriers,
wherein the composition treats bone osteonecrosis.

12. The composition of claim 11, wherein the growth promoting agent or macrophage depletion reagent is selected from bone morphogenic protein 2 (BMP-2), bone morphogenic protein 7 (BMP-7), Vascular endothelial growth factor (VEGF), clodronate, or Clodrosome.

13. The composition of claim 11, wherein the composition is a biocompatible isotonic fluid and optionally comprises biocompatible detergents, biocompatible surfactants, biocompatible alcohols, antibiotics, preservatives, or combinations thereof, wherein the washing fluid cleans at least 50, 60, 70, 75, 80, 85, or 90% of the volume within the bone.

14. The composition of claim 11, further comprising at least one of autologous bone marrow, autologous bone stem cells, bone graft material, bone void filler, cancellous bone graft or fragments, osteoconductive material, osteoproliferative material, osteoinductive material, or bone material infused collagen matrix.

15. The composition of claim 11, wherein the GT is a Gelatin tyramine hydrogel, a gelatin-heparin tyramine (GHT) hydrogel, or both, which comprises a trace amount of hydrogen peroxide (0.1 mM-10 mM), a horseradish peroxidase enzyme (0.1-10 unit/ml), wherein the bone growth promoting agent or a macrophage depletion reagent retains at least 80% bioactivity, and optionally comprising heparin at (0.5-5% w/v) that is formed ex vivo or in situ.

16. The composition of claim 11, wherein the GelMa is a Gelatin methacryloyl (GelMa) hydrogel, a gelatin acryloyl (GelAC) hydrogel, or both, which comprises a photoinitiator at (0.01-1% w/v) and the hydrogel is formed by ultraviolet light ex vivo or in situ.

17. The composition of claim 11, wherein the collagen is a collagen hydrogel (0.3-3%, w/v) formed by gelation at 37° C. without the addition of crosslinkers that is formed ex vivo or in situ.

18. The composition of claim 11, wherein the hydrogel forming peptide is synthetic peptide-RADA16 (0.25%-2.5%, w/v) is a thermosensitive hydrogel that is formed ex vivo or in situ.

19. The composition of claim 11, wherein the composition is adapted to inject into a femoral head, a humeral head, a knee condyle, a proximal tibia, or an ankle talus.

20. The composition of claim 11, wherein the composition is adapted for injection into an osteonecrosis is Legg-Calvé-Perthes Disease, idiopathic (unknown cause), due to corticosteroid, trauma, alcohol, sickle cell disease, or other known causes of osteonecrosis.

21. A method of making a composition for injection into osteonecrotic tissue comprising:
mixing at least one of a Gelatin tyramine (GT), a gelatin-heparin tyramine (GHT), a gelatin methacryloyl (GelMa), a gelatin acryloyl (GelAC), collagen, or a hydrogel-forming peptide in an amount sufficient to form a hydrogel, and one or more bone growth promoting agents or macrophage depletion reagents in an amount effective to induce bone growth in a femoral head, a humeral head, a knee condyle, a proximal tibia, or an ankle talus; and
optionally one or more pharmaceutically acceptable carriers,
wherein the composition treats bone osteonecrosis.

22. The method of claim 21, wherein the growth promoting agent or macrophage depletion reagent is selected from bone morphogenic protein 2 (BMP-2), bone morphogenic protein 7 (BMP-7), Vascular endothelial growth factor (VEGF), clodronate, or Clodrosome.

23. The method of claim 21, wherein the composition is a biocompatible isotonic fluid and optionally comprises biocompatible detergents, biocompatible surfactants, biocompatible alcohols, antibiotics, preservatives, or combinations thereof, wherein the washing fluid cleans at least 50, 60, 70, 75, 80, 85, or 90% of the volume within the bone.

24. The method of claim 21, further comprising at least one of autologous bone marrow, autologous bone stem cells, bone graft material, bone void filler, cancellous bone graft or fragments, osteoconductive material, osteoproliferative material, osteoinductive material, or bone material infused collagen matrix.

25. The method of claim 21, wherein the GT is a Gelatin tyramine hydrogel, a gelatin-heparin tyramine (GHT) hydrogel, or both, which comprises a trace amount of hydrogen peroxide (0.1 mM-10 mM), a horseradish peroxidase enzyme (0.1-10 unit/ml), wherein the bone growth promoting agent or a macrophage depletion reagent retains at least 80% bioactivity, and optionally comprising heparin at (0.5-5% w/v) that is formed ex vivo or in situ.

26. The method of claim 21, wherein the GelMa is a Gelatin methacryloyl (GelMa) hydrogel, a gelatin acryloyl (GelAC) hydrogel, or both, which comprises a photoinitiator at (0.01-1% w/v) and the hydrogel is formed by ultraviolet light ex vivo or in situ.

27. The method of claim 21, wherein the collagen is a collagen hydrogel (0.3-3%, w/v) formed by gelation at 37° C. without the addition of crosslinkers that is formed ex vivo or in situ.

28. The method of claim 21, wherein the hydrogel-forming peptide is synthetic peptide-RADA16 (0.25%-2.5%, w/v) is a thermosensitive hydrogel that is formed ex vivo or in situ.
